(19) United States
(12) Patent Application Publication
(10) Pub. No.: US 2007/0276307 A1
(43) Pub. Date: Nov. 29, 2007

(54) ADAPTABLE COMPRESSION ORTHOSIS

(76) Inventor: Jeffrey Erenstone, Fitchburg, MA (US)

Correspondence Address: NELSON & NELSON, P.C., 2984 E. EVERGREEN AVE., SLC, UT, 84109, US

(21) Appl. No.: 11/752,404

(22) Filed: May 23, 2007

Related U.S. Application Data
(60) Provisional application No. 60/803,222, filed on May 25, 2006.

(51) Int. Cl. A61F 5/00

(52) U.S. Cl. 602/27

(57) ABSTRACT

A highly adaptable compression apparatus to apply a compression force to an extremity of a user. The apparatus includes a removable compression overlay to selectively adhere to a compression sheath surrounding an extremity of a user. A plurality of independently adjustable straps may be coupled to the compression overlay. Tightening the straps may contract the compression overlay to tighten the compression sheath around the extremity, thereby increasing a hydrostatic compression force imparted thereto

### RELATED APPLICATIONS

This application claims priority to U.S. Provisional Patent App. No. 60/803,222, filed on May 25, 2006, and entitled CUSTOMIZABLE COMPRESSION ORTHOSIS.

### BACKGROUND

1. Field of the Invention

This invention relates to orthopedic devices, and more particularly to an orthopedic compression brace to apply a hydrostatic compression force to an extremity of a user.

2. Background

Functional fracture bracing is premised on the engineering principle of hydrostatic compression. Specifically, compressing the soft tissue around the bone produces increased intra-vascular hydrostatic pressure to stabilize the fracture and promote healing. The increased hydrostatic pressure created by the brace shifts the load that would be borne in the bone to the surrounding soft tissue, such that the soft tissue has a greater load-bearing effect than the brace itself. A rigid exterior frame may also be implemented to further stabilize the injury.

Fracture orthoses are advantageous over traditional rigid casting in many ways. Particularly, fracture orthoses are easily applied by a skilled physician or other professional. The speed of application can be a great asset in the trauma setting, as it enables the physician or other professional to manage multiple patients and perform any necessary adjustments quickly and easily. Fracture orthoses may also be easily adjusted to accommodate changes in the volume of the fractured extremity over time. In this manner, fracture orthoses may promote healing by maintaining hydrostatic pressure to stabilize the fractured bone as the volume of the affected limb decreases due to atrophy, or increases due to swelling. Fracture orthoses are also readily removable to enable effective hygiene management, and are generally more economic than traditional casts.

Many known fracture orthoses, however, are disadvantageous to patients that would benefit from light to moderate activity, such as stress fracture patients. Indeed, such fracture orthoses are generally too cumbersome and rigid to permit the movement required for such activity. Accordingly, recent developments in stress fracture treatment include stirrup-style pneumatic ankle control braces. Even these braces, however, present certain drawbacks.

Specifically, some stress fracture orthoses exert medial-lateral compression force to effectuate increased hydrostatic pressure, and are thus less effective in stabilizing a fracture than orthoses that provide circumferential compression force. Known stress fracture orthoses are also generally bulky and may be awkward to wear. For example, as previously mentioned, some lower leg orthoses require a stirrup underneath the foot that may create discomfort.

Also, known stress fracture orthoses, as well as fracture orthoses generally, are size-specific. This feature necessitates manufacture and storage of substantial quantities of orthoses in a wide range of sizes—some of which may never be used. Similarly, known orthoses are also generally specific to the right and left sides of a user's body, further contributing to an oversupply of orthoses and, inevitably, waste.

Accordingly, what is needed is a highly effective orthosis that exerts circumferential compressive force to effectuate increased hydrostatic pressure to stabilize a fracture. Further what is needed is an orthosis that is comfortable to wear and permits light to moderate activity. Also what is needed is an orthosis that is compact to store. Finally what is needed is a customizable orthosis that may be sized to a particular user, implemented on either a right or left extremity, and that substantially conforms to the user's body.

Such an orthosis is disclosed and claimed herein.

### SUMMARY

The present invention has been developed in response to the present state of the art, and in particular, in response to the problems and needs in the art that have not yet been met for applying a hydrostatic compression force to an extremity of a user. Accordingly, the present invention has been developed to provide an apparatus, system and method for applying a compression force to an extremity of a user that overcomes many or all of the above-discussed shortcomings in the art.

A highly adaptable compression apparatus to apply a compression force to an extremity of a user in accordance with the present invention may include a compression overlay having a cinching mechanism coupled thereto. The compression overlay may selectively adhere to a compression sheath substantially surrounding an extremity of a user. The cinching mechanism may be tightened to contract the compression overlay, thereby tightening the compression sheath around the extremity. In this manner, the present invention may increase a hydrostatic compression force imparted to the extremity.

In some embodiments, the compression overlay includes more than one elongated mounting panel to mount the compression overlay to the compression sheath. A distance between the mounting panels may be adjusted to accommodate various users. In one embodiment, the mounting panels include hook and loop-type fastening elements to mount the compression overlay to the compression sheath. Further, one or more substantially rigid stays may be longitudinally integrated into the compression overlay to increase stability of the extremity.

In certain embodiments, the cinching mechanism of the present invention includes a cam-over device, such as straps or laces. In one embodiment, the cam-over device includes a plurality of straps. Each strap includes a primary looped portion fixed to the compression overlay, and a secondary portion slidably connected to the primary looped portion. The primary looped portion may be fixed to one mounting panel of the compression overlay and slidably connected to another mounting panel. In some embodiments, the secondary portion removably attaches the secondary portion to the compression sheath.

A system of the present invention is also presented to apply a compression force to an extremity of a user. The system may include a unitary compression sheath to substantially surround an extremity of a user. The unitary compression sheath may include a resilient portion to apply a hydrostatic compression force to the extremity, and an elastic portion to facilitate application and removal of the compression sheath. The system may further include a removable compression overlay to selectively adhere to the compression sheath. A cinching mechanism coupled to the overlay may contract the compression overlay upon tightening, thereby tightening the compression sheath around the extremity and increasing a hydrostatic compression force imparted thereto.

In some embodiments, the compression sheath may include longitudinal seams connecting the resilient portion to the elastic portion. These seams may facilitate positioning of the compression sheath with respect to the extremity. Longitudinal folds may extend from the longitudinal seams and be adapted to reduce pressure and irritation over bony prominences of the extremity.

A method to apply a compression force to an extremity of a user may include positioning a compression sheath around an extremity of a user and applying a removable compression overlay to the compression sheath. A cinching mechanism coupled to the compression overlay may be tightened to contract the compression overlay, thereby tightening the compression sheath around the extremity. The cinching mechanism may then be secured to increase a hydrostatic compression force imparted to the extremity. Further, in some embodiments, substantially rigid stays integrated into the compression overlay may be selectively positioned to increase stability of the extremity.

In one embodiment, applying the removable compression overlay may include mounting a first mounting panel of the overlay to the compression overlay. A second mounting panel of the overlay may be adjusted relative to the first mounting panel as needed to accommodate a particular extremity. The second mounting panel may then be mounted to the compression sheath.

These and other features and advantages of the present invention will be set forth or will become more fully apparent in the description that follows. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above-recited and other advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 is a front view of one embodiment of an adaptable compression orthosis in accordance with the present invention;

FIG. 2 is a perspective view of one embodiment of an adaptable compression orthosis applied to a lower leg of a user in accordance with the present invention;

FIG. 3 is a perspective view of an alternative embodiment of an adaptable compression orthosis in accordance with the present invention;

FIG. 4 is a front view of one embodiment of a compression sheath that may be used in conjunction with an adapatable compression orthosis in accordance with the present invention;

FIG. 5 is a perspective view of the compression sheath of FIG. 4 fitted to a lower leg of a user; and

FIG. 6 is a flow chart detailing steps for applying a compression force to an extremity of a user in accordance with embodiments of the present invention.

### DETAILED DESCRIPTION OF THE INVENTION

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The presently preferred embodiments of the invention will be best understood by reference to the drawings wherein like parts are designated by like numerals throughout.

As used in this specification, the term “orthosis” refers to an externally applied device used to modify the structural and/or functional characteristics of a limb or other extremity. The term “user” refers to any human or animal utilizing an orthosis to stabilize an extremity or other portion of the body as described herein.

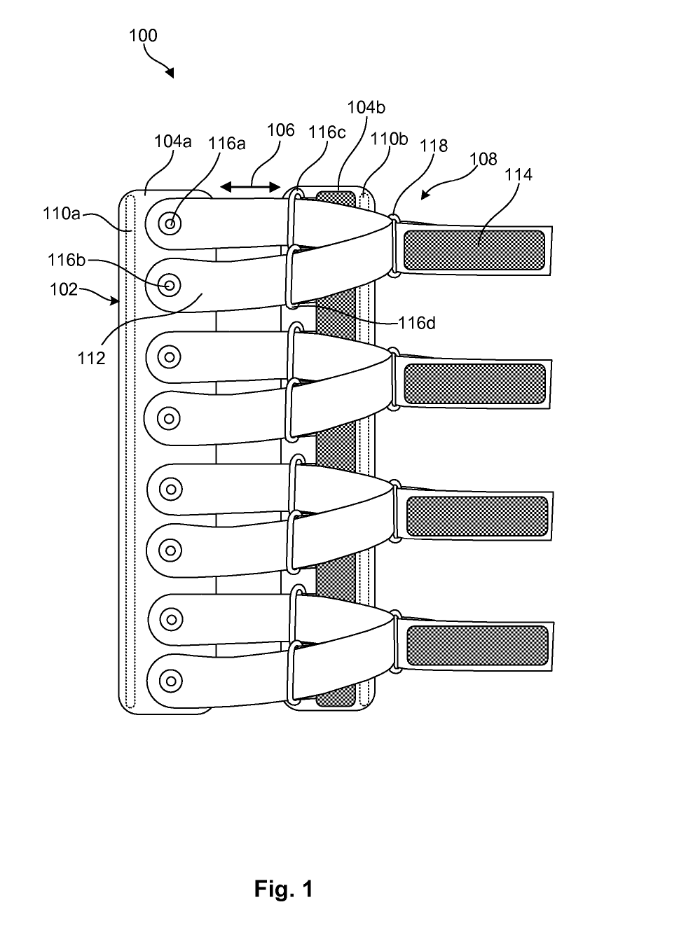

Referring now to FIG. 1, an adaptable compression orthosis 100 in accordance with the present invention may comprise a compression overlay 102 to selectively adhere to a compression sheath (not shown). The compression overlay 102 may be unitary or modular in design and may include one or more mounting panels 104 to mount the compression overlay 102 to the compression sheath. In one embodiment, for example, the mounting panels 104 include a hook and loop fastener, such as Velcro®, to mount the compression overlay 102 to the compression sheath.

A distance 106 between the mounting panels 104 may be adjusted as needed to fit a particular user. For example, the distance 106 may be shortened to accommodate an extremity having a larger circumference, and lengthened to accommodate an extremity having a smaller circumference. The distance 106 between the mounting panels 104 may also be adjusted along a length of the compression overlay 102 to accommodate circumferential variances along an extremity. For example, in a lower leg application, the distance 106 between mounting panels 104 may be shortened at the widest part of the calf and lengthened towards the ankle. This adjustability may also enable the compression overlay 102 of the present invention to be used in connection with various compression sheaths having varying topographies and contours.

The compression overlay 102 may further include a cinching mechanism 108 attached to the mounting panels 104. The cinching mechanism 108 may include a cam-over device such as straps, laces, or any other such device known to those in the art. In operation, the cinching mechanism 108 may contract the compression overlay 102 upon tightening, thereby tightening the compression sheath around an extremity of a user. As a result, the hydrostatic compression force imparted to the extremity may be increased.

In one embodiment, the cinching mechanism 108 includes straps having a primary looped portion 112 fixed to the compression overlay 102, and a secondary portion 114 slidably connected to the primary looped portion 112. Alternatively, each strap may include a unitary or other modular design. The straps may include any natural or synthetic material known to those in the art. The primary looped portion 112 may be fixed to the compression overlay 102 by a fastening device 116 such as a ring, a clasp, a seam, a snap, a rivet, or the like. The secondary portion 114 may be slidably connected to the primary looped portion 112 via a connecting device 118, such as a slidable ring.

In one embodiment, the primary looped portion 112 is attached to multiple mounting panels 104a, 104b of the compression overlay 102. The primary looped portion 112 may be fixedly connected to one mounting panel 104a with rivets 116a, 116b, and slidably connected to another mounting panel 104b with rings 116c, 116d to facilitate tightening the cam-over device 108 to contract the compression overlay 102. In one embodiment, the fastening devices 116c, 116d used to attach the primary looped portion 112 to the second mounting panel 114b are flexibly attached to the second mounting panel 114b to accommodate minor circumferential variation due to muscle flexion and the like.

The secondary portion 114 may include a removable fastening device (not shown) to secure the cam-over device 108 relative to the compression sheath, thereby maintaining the hydrostatic compression force imparted to the extremity. In one embodiment, for example, the secondary portion 114 may include a hook and loop fastening device, such as Velcro®, to secure the cam-over device 108 relative to the compression sheath. Alternatively, the secondary portion 114 may include a buckle, a snap, a hook, a loop, or any other device known to those in the art to secure the cam-over device 108 relative to the compression sheath.

Removably securing the cam-over device 108 relative to the compression sheath may enable customized application of the hydrostatic compression force to the extremity. Moreover, in some embodiments, variable distance between mounting panels 104 and adjustable securement of the cam-over device 108 with respect to the compression sheath combine to enable the present invention to provide a uniquely high level of adaptability.

In some embodiments, the compression overlay 102 may further include one or more substantially rigid stays 110 to further stabilize an affected extremity. The stays 110 may be longitudinally integrated into one or more of the mounting panels 104 to provide increased load-bearing support while avoiding interference with the adjustability and function of the present invention.

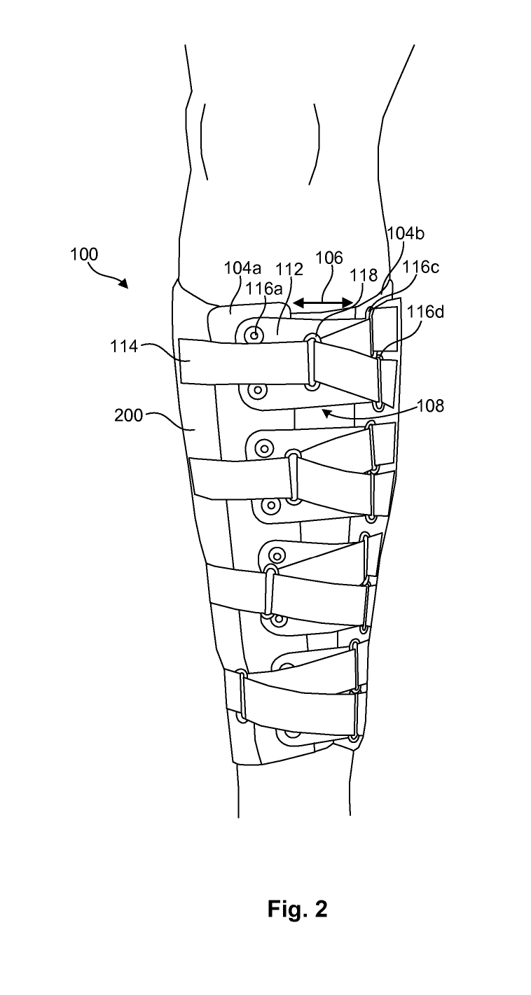

Referring now to FIG. 2, the compression overlay 102 may be selectively attached to a compression sheath 200 and oriented to facilitate a user's ability to tighten the cam-over device 108 to apply a desired amount of hydrostatic compression force to an extremity. Moreover, in embodiments where the compression overlay 102 is entirely removable, an adaptable compression orthosis 100 in accordance with the present invention may not be side-specific, but may be implemented on either a right or left extremity.

In one embodiment, for example, the compression overlay 102 may be mounted to the compression sheath 200 and situated at the front of a user's lower leg. The user may mount one mounting panel 104a substantially parallel to the tibia, or shin bone, and adjust a distance 106 between the mounting panel 104a and a second mounting panel 104b as needed to accommodate the lower leg. In some embodiments, the first mounting panel 104a may be substantially permanently attached to the compression sheath 200, while the second mounting panel 104b may be removably attached thereto.

Upon securing the second mounting panel 104b to the compression sheath 200, the user may grasp the secondary portion 114 of the cam-over device 108 and pull it laterally towards the first mounting panel 104a. In this manner, the secondary portion 114 of the cam-over device 108 may reverse over the primary looped portion 112 to cinch the mounting panels 104a, 104b together, thereby contracting the compression overlay 102.

The secondary portion 114 of the cam-over device 108 may be removably secured to the compression sheath 200 by way of a hook and loop fastening device, such as Velcro®, or by any other means known to those in the art. Alternatively, the cinching mechanism 108 may be secured to the compression overlay 102, and/or may be secured by an independent securing device. An independent securing device may include, for example, a knot, a bow, a clasp, a cinch, or any other suitable securing device known to those in the art. In this manner, the user may tighten and secure the compression sheath 200 around the lower leg to increase a hydrostatic compression force imparted thereto.

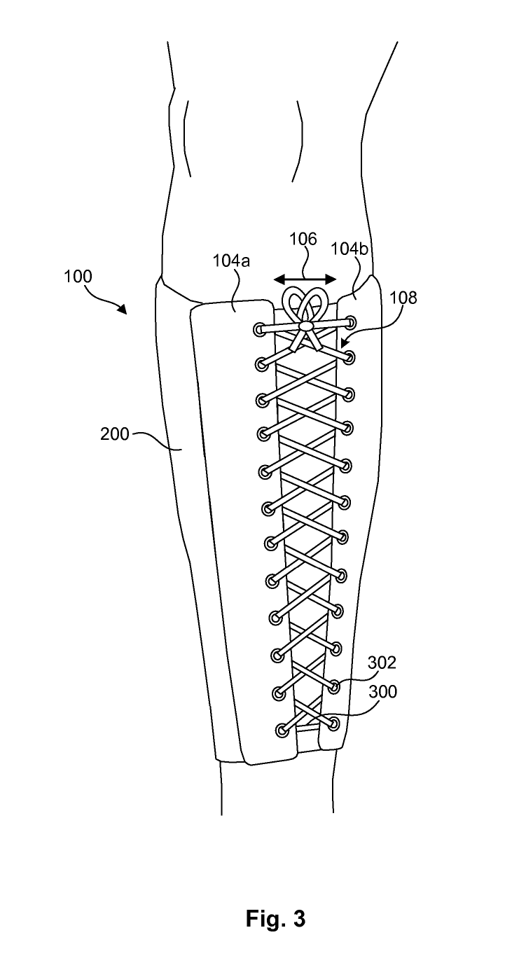

Referring now to FIG. 3, an alternative embodiment of the cinching mechanism 108 may include laces 300 extending between opposing sides of the compression overlay 102. In one embodiment, the laces 300 may extend between two mounting panels 104a, 104b. Specifically, each mounting panel 104 may include multiple eyelets 302 through which the laces 300 are threaded. Tightening the laces 300 may cinch the mounting panels 104 together, thereby contracting the compression overlay 102 to tighten the compression sheath 102 around an affected extremity. The laces 300 may be secured by a knot, a bow, a clasp, or any other means known to those in the art.

In some embodiments, as mentioned above, the laces 300 or other cam-over device 108 may cooperate with multiple mounting panels 104 to facilitate a customized application of hydrostatic compression force to an extremity. Indeed, while a distance 106 between mounting panels 104 may be adjustable, a user may be limited in his ability to manually increase this distance 106 to tighten the compression sheath 200 around the extremity. Accordingly, the laces 300, straps, or other cam-over device 108 provide additional leverage to enable the user to easily and effectively tighten the compression sheath 200 around the extremity to increase the amount of hydrostatic compression force imparted thereto.

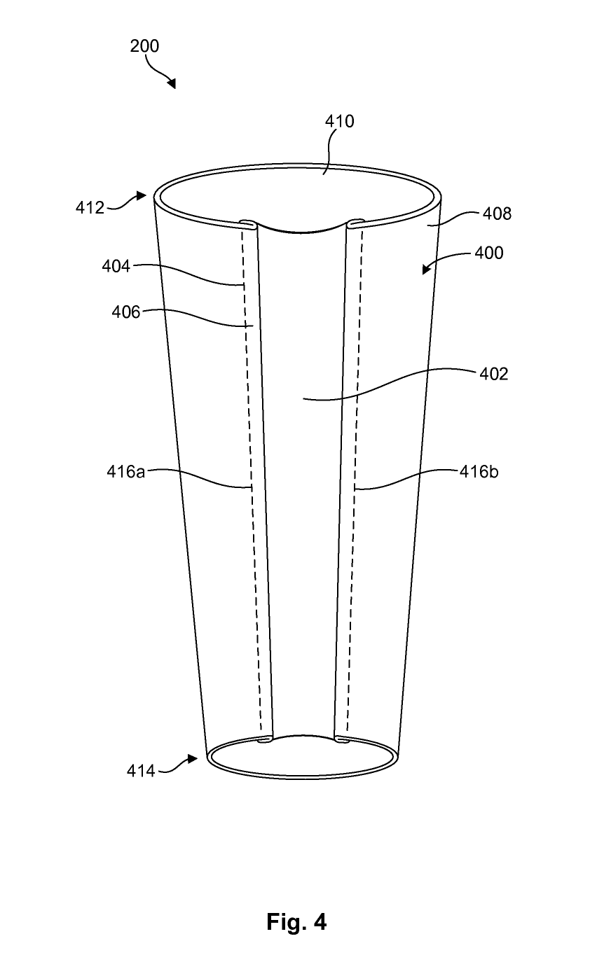

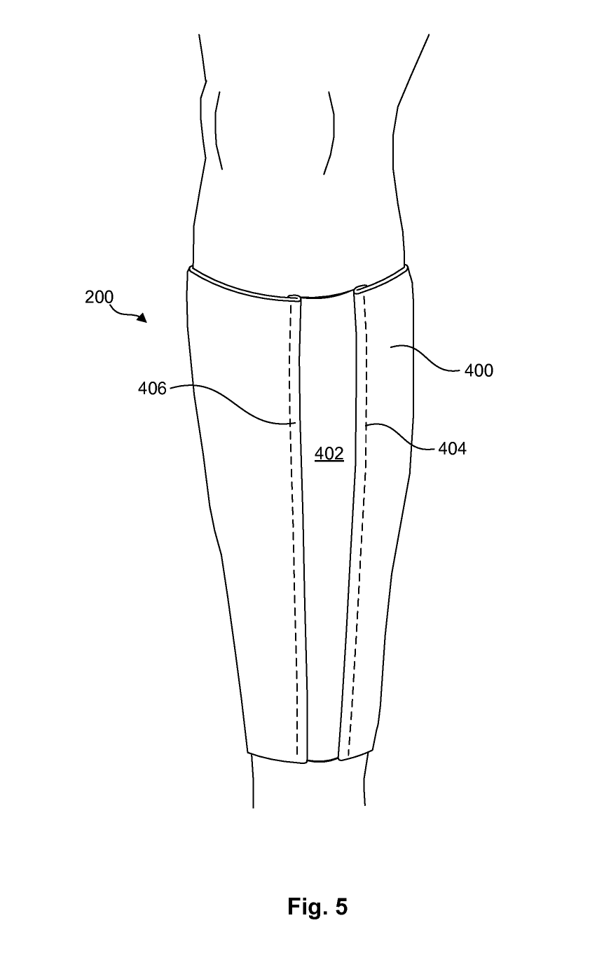

Referring now to FIGS. 4 and 5, one embodiment of an adaptable compression orthosis 100 in accordance with the present invention includes a unitary compression sheath 200 to substantially surround an extremity of a user. The unitary compression sheath 200 may include a resilient portion 400 and an elastic portion 402. The resilient portion 400 may apply a hydrostatic compression force to the extremity, while the elastic portion 402 may facilitate application and removal of the compression sheath 200.

The resilient portion 400 may be adapted to substantially evenly distribute a circumferential compression force to stabilize an extremity. In some embodiments, the resilient portion 400 may include a flexible, substantially resilient material such as Neoprene®, foam rubber, plastic, nylon, or other suitable material known to those in the art. The resilient portion 400 may further include multiple layers to facilitate application and maintenance of a hydrostatic compression force.

In one embodiment, for example, the resilient portion 400 includes an outer layer 408 including a hook and loop fastening device such as Velcro®. This may enable the outer layer 408 to cooperate with the secondary portion 114 of the cinching mechanism 108 to secure the same. The resilient portion 400 may further include an inner layer 410 to mediate contact between the resilient portion 400 and the user's skin. The inner layer 410 may include a breathable material to improve circulation, reduce perspiration, and/or increase overall comfort. In some embodiments, the resilient portion 400 may further include one or more seams (not shown) and/or substantially collapsible sections to facilitate compact storage.

The elastic portion 402 of the compression sheath 200 may enable the compression sheath 200 to be applied over a foot, hand or other intermediary appendage of a user to substantially surround an affected extremity. In certain embodiments, for example, the elastic portion 402 may include Lycra®, Spandex®, nylon, or any other suitable elastic material known to those in the art.

In some embodiments, the elastic portion 402 may be attached to the resilient portion 400 via longitudinal seams 404 extending from a top 412 to a bottom 414 of the compression sheath 200. In one embodiment, the seams 404 are integrated to create longitudinal folds 406 in the resilient portion 400 that extend beyond the edges 416a, 416b of the elastic portion 402.

The longitudinal folds 406 may be positioned substantially laterally of a bone or bony prominence to reduce friction and pressure over the same. In this manner, the longitudinal folds 406 may bridge an applied hydrostatic compression force over sensitive areas to avoid skin irritation and pressure, while nevertheless imparting a substantially circumferential compression force to stabilize an affected extremity.

In some embodiments, the compression sheath 200 may include dimensions sufficient to accommodate a wide range of users. For example, in one embodiment, a compression sheath 200 for lower leg use in accordance with the invention includes small, medium narrow, medium wide, large narrow, and large wide sizes. These sizes are adapted to accommodate various users depending on lower leg length and calf circumference.

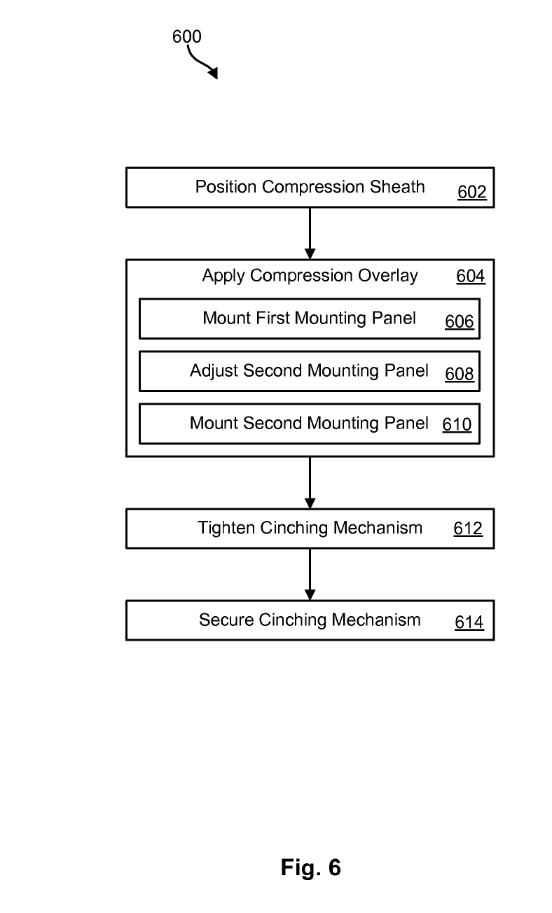

Referring now to FIG. 6, a method 600 to apply a compression force to an extremity of a user in accordance with the present invention may include positioning 602 a compression sheath to substantially surround an extremity of a user and applying 604 a removable compression overlay to the compression sheath.

In some embodiments, the compression panel may include more than one mounting panel. A first mounting panel may be removably attached 606 to the compression sheath, or may be substantially permanently attached 606 thereto. A second mounting panel may be adjusted 608 relative to the first mounting panel as needed to accommodate an affected extremity of a particular user. The second mounting panel may then be removably attached 610 to the compression sheath.

A cinching mechanism attached to the compression overlay maybe tightened 612 to contract the compression overlay, thereby tightening the compression sheath around the extremity. The cinching mechanism may then be secured 614 to increase a hydrostatic compression force imparted to the extremity.

In some embodiments, the cinching mechanism may be secured to the compression sheath. Alternatively, the cinching mechanism may be secured to the compression overlay, and/or may be independently secured by a securing device. A securing device may include, for example, a knot, a bow, a clasp, a cinch, or any other suitable securing device known to those in the art.

In some embodiments, a method 600 in accordance with the present invention may further include positioning (not shown) a substantially rigid stay of the compression overlay to increase stability of the extremity. Specifically, one or more substantially rigid stays may be positioned to increase load-bearing support with respect to the affected extremity.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A highly adaptable compression apparatus to apply a compression force to an extremity of a user, the apparatus comprising:
a removable compression overlay to selectively adhere to a compression sheath substantially surrounding an extremity of a user; and
a cinching mechanism coupled to the compression overlay and configured to contract the compression overlay upon tightening by the user, thereby tightening the compression sheath around the extremity and increasing a hydrostatic compression force imparted thereto.

2. The highly adaptable compression apparatus of claim 1, wherein the compression overlay comprises more than one elongated mounting panel adapted to mount the compression overlay to the compression sheath.

3. The highly adaptable compression apparatus of claim 2, wherein a distance between the more than one elongated mounting panels is adjustable to accommodate various users.

4. The highly adaptable compression apparatus of claim 2, wherein the more than one elongated mounting panel comprises a hook and loop fastening element to mount the compression overlay to the compression sheath.

5. The highly adaptable compression apparatus of claim 1, further comprising at least one substantially rigid stay longitudinally integrated into the compression overlay to increase stability of the extremity.

6. The highly adaptable compression apparatus of claim 1, wherein the cinching mechanism comprises a cam-over device.

7. The highly adaptable compression apparatus of claim 6, wherein the cam-over device is selected from the group consisting of straps and laces.

8. The highly adaptable compression apparatus of claim 6, wherein the cam-over device comprises a plurality of straps, each of the plurality of straps comprising:
a primary looped portion fixed to the compression overlay; and
a secondary portion slidably connected to the primary looped portion.

9. The highly adaptable compression apparatus of claim 8, wherein the primary looped portion is fixed to a first mounting panel of the compression overlay and slidably attached to a second mounting panel of the compression overlay.

10. The highly adaptable compression apparatus of claim 8, wherein the secondary portion removably attaches to the compression sheath.

11. A system to apply a compression force to an extremity of a user, the system comprising:
a unitary compression sheath adapted to substantially surround an extremity of a user, the compression sheath having a resilient portion to apply a hydrostatic compression force to the extremity and an elastic portion to facilitate application and removal of the compression sheath;
a removable compression overlay to selectively adhere to the compression sheath; and
a cinching mechanism coupled to the compression overlay and configured to contract the compression overlay upon tightening by the user, thereby tightening the compression sheath around the extremity and increasing a hydrostatic compression force imparted thereto.

12. The system of claim 11, wherein the compression sheath further comprises longitudinal seams connecting the resilient portion to the elastic portion.

13. The system of claim 12, further comprising longitudinal folds extending from longitudinal seams, wherein the longitudinal folds are adapted to reduce pressure and irritation over a bony prominence of the extremity.

14. The system of claim 11, wherein the compression overlay comprises more than one elongated mounting panel adapted to mount the compression overlay to the compression sheath.

15. The system of claim 14, wherein a distance between the more than one elongated mounting panels is adjustable to accommodate various users.

16. The system of claim 10, wherein the compression overlay comprises at least one substantially rigid stay longitudinally integrated therein to increase stability of the extremity.

17. The system of claim 11, wherein the cinching mechanism comprises a plurality of straps, each of the plurality of straps comprising:
a primary looped portion fixed to the compression overlay; and
a secondary portion slidably connected to the primary looped portion.

18. A method to apply a compression force to an extremity of a user, the method comprising:
positioning a compression sheath to substantially surround an extremity of a user;
applying a removable compression overlay to the compression sheath;
tightening a cinching mechanism attached to the compression overlay to contract the compression overlay and tighten the compression sheath around the extremity; and
securing the cinching mechanism to increase a hydrostatic compression force imparted to the extremity.

19. The method of claim 18, wherein applying a removable compression overlay comprises:
mounting a first mounting panel of the removable compression overlay to the compression sheath;
adjusting a second mounting panel of the removable compression overlay relative to the first mounting panel to accommodate the extremity; and
mounting the second mounting panel to the compression sheath.

20. The method of claim 18, further comprising positioning a substantially rigid stay of the compression overlay to increase stability of the extremity.
